(19) United States
(12) Patent Application Publication
(10) Pub. No.: US 2002/0182587 A1
(43) Pub. Date: Dec. 5, 2002

(54) Methods and compositions for the treatment and prevention of smooth muscle cell proliferation

(76) Inventor: Sivaram Pillarisetti, Norcross, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/091,357

(22) Filed: Mar. 1, 2002

Related U.S. Application Data
(60) Provisional application No. 60/272,951, filed on Mar. 2, 2001.

(51) Int. Cl. C12Q 1/00; C12Q 1/68; G01N 33/53; G01N 33/567; A61K 39/395; A61K 31/70; A61K 48/00; A61K 38/17

(52) U.S. Cl. 435/4; 435/6; 435/7.2; 514/12; 424/155.1; 514/44; 514/23

(57) ABSTRACT

Methods and Compositions for the Treatment and Prevention of Smooth Muscle Cell Proliferation The present invention is directed to methods and compositions for diagnosing, treating and preventing smooth muscle cell proliferation and hyperplasia, including vascular occlusive conditions. Such conditions include, but are not limited to, thrombosis, atherosclerosis and restenosis after angioplasty. Preferred embodiments comprise methods and compositions that effect HSPGs associated with smooth muscle cell proliferation and more preferably, effect perlecan.

### RELATED APPLICATIONS

This application claims benefit of U.S. Provisional Application No. 60/272,951, filed Mar. 2, 2001.

### TECHNICAL FIELD

The present invention relates to compositions and methods for identifying compounds that effect vascular conditions related to smooth muscle cell proliferation. In particular, the present invention comprises methods and compositions for treating vascular occlusive pathologies, and preferably comprises compounds that inhibit smooth muscle cell proliferation through the induction of heparan sulfate proteoglycans.

### BACKGROUND OF THE INVENTION

Abnormal vascular smooth muscle cell (SMC) proliferation is thought to contribute to the pathogenesis of vascular occlusive lesions, including arteriosclerosis, atherosclerosis, restenosis, and graft atherosclerosis after coronary transplantation. Vascular SMC proliferation is a common consequence of endothelial injury and is believed to be an early pathogenetic event in the formation of atherosclerotic plaques. Many humans and animals have limited lifespans and lifestyles because of such conditions.

Percutaneous coronary artery intervention (PTCA) procedures are the most common in-patient hospital procedure in the United States. According to the American Heart Association, about one-third of the patients that undergo balloon angioplasty have restenosis of the widened segment within approximately 6 months. It may be necessary to perform another angioplasty or coronary artery bypass surgery on restenosed arteries. A key feature of restenosis is an injury response that results in activation of an inflammatory cascade and remodeling of the cells both inside and outside the carotid artery wall. This includes excessive growth of connective tissue and smooth muscle into the lumen of the artery known as neointimal hyperplasia. Currently there are no effective pharmacological treatments available that control the pathogenesis of vascular occlusive lesions, such as, but not limited to, arteriosclerosis, atherosclerosis, restenosis, and graft atherosclerosis after coronary transplantation. Identification of effective therapeutics with minimal side effects will restore quality of life without requiring additional surgical procedures such as coronary artery bypass surgery.

The mechanisms involved in the control of vascular conditions related to SMC function are not clear and there is no preventive therapy against SMC activation. Thus, what is needed are methods and compositions for treatment and prevention of vascular occlusive conditions. In particular, what is needed are methods and compositions to prevent and treat restenosis following treatments of vascular tissues.

What is also needed are methods and compositions for the alteration of gene expression in arterial wall cells to inhibit thrombosis and SMC proliferation. In particular what is needed are methods and compositions that inhibit SMC proliferation and related intimal hyperplasia.

### SUMMARY OF THE INVENTION

The present invention comprises methods and compositions for prevention and treatment of vascular occlusive conditions including, but not limited to, neointimal hyperplasia. Preferred embodiments of the present invention include methods and compositions for measuring the induction of HSPG including, but not limited to, syndecan, glypican and perlecan. The present invention additionally comprises assays for rapid and accurate high throughput screening of molecules that induce HSPG synthesis. Compositions comprising the molecules and compounds identified are used in methods for effective therapies to treat vascular occlusive pathologies related to smooth muscle cell proliferation.

The present invention also comprises compositions comprising the compounds having a desired activity identified by the assays. The compositions have utility in treatment of cells, tissues or whole organisms. Such compositions are formulated for administration in an effective amount for treatment of conditions related to smooth muscle cell proliferation such as biological conditions including, atherosclerosis, restenosis, transplant vasculopathy, cardiac allograft vasculopathy and graft atherosclerosis after coronary transplantation. The present invention additionally comprises methods and compositions that are effective in mediating the clinically deleterious effects of syndecan, glypican and perlecan and other HSPGs. Such methods and compositions are used to regulate vascular SMC replication, migration, gene expression and differentiation. In particular, such methods and compositions comprise compositions and methods for regulation of perlecan for prevention and treatment of vascular conditions. A most preferred embodiment comprises compositions and methods for induction of perlecan synthesis and expression for the treatment and prevention of restenosis of vascular tissues. The compositions may comprise other active compounds or pharmaceutical adjuncts that are needed for administration of the compound or compounds with the desired activity.

### BRIEF DESCRIPTION OF THE FIGURES

FIG. 1 A and B are graphs showing measurements of proteoglycans in cells which are grown under serum-free conditions and control cells grown with serum.

FIG. 2 is a graph showing perlecan inhibition of SMC proliferation.

FIG. 3 is a graph showing DNA synthesis effects due to perlecan.

FIG. 4 is a graph showing cellular quiescence in the presence of perlecan.

FIG. 5 is a graph showing the effects of perlecan with growth inhibitors.

FIG. 6 A is a graph showing the average of IA/MA ratios for each animal.

FIG. 6 B is a graph showing the average of IA/MA ratios of all sections regardless of the number of animals.

### DETAILED DESCRIPTION OF THE INVENTION

The present invention comprises methods and compositions for detecting compounds or molecules that have specific biological effects and that may be useful as therapeutic agents. The present invention also comprises methods and compositions for the treatment and prevention of vascular occlusive conditions such as, but not limited to neointimal hyperplasia, restenosis, transplant vasculopathy, cardiac allograft vasculopathy, atherosclerosis, and arteriosclerosis. Such methods and compositions comprise methods for inhibition of smooth muscle cell (SMC) growth and proliferation, and for induction of quiescence in smooth muscle cells. Preferred embodiments of the present invention comprise methods and compositions for inducing heparan sulfate proteoglycan (HSPG) synthesis and expression, including, but not limited to, the induction of HSPGs such as syndecan, glypican and perlecan, and most preferably perlecan synthesis and gene expression.

Neointimal hyperplasia is commonly seen after various forms of vascular injury and a major component of the vein graft's response to harvest and surgical implantation into high-pressure arterial circulation. In neointimal hyperplasia, smooth muscle cells in the middle layer of the vessel wall become activated, divide, proliferate and migrate into the inner layer. The resulting abnormal neointimal cells express pro-inflammatory molecules, including cytokines, chemokines and adhesion molecules that further trigger a cascade of events that lead to occlusive neointimal disease and eventually graft failure.

Proliferation of SMC in response to local injury is a major feature of vascular proliferative disorders such as atherosclerosis and restenosis after angioplasty. Though not wishing to be bound to any particular theory, it is generally believed that the endothelium regulates the growth of the underlying SMC. In normal vessels, SMC are quiescent, but they proliferate when damage to the endothelium occurs. The endothelium, in addition to producing a variety of growth factors, also generates key growth inhibitors. HSPGs are components of vascular cell membranes and extracellular matrix that are believed to control a variety of vascular functions including functioning as a barrier against cationic molecules and macromolecules, protecting the main structural component of the basement membrane, type IV collagen, from proteolytic attack, binding cytokines and growth factors including, but not limited to, basic fibroblast growth factor (bFGF), vascular endothelial growth factor (VEGF), hepatocyte growth factor (HGF), keratinocyte growth factor (KGF), and transforming growth factor β (TGF-β), functioning as storage for these cytokines, regulating mesodermal cell fate, positioning of the heart, acting in vasculogenesis and angiogenesis after ischemic injury, effecting interactions of cells with adhesive proteins and blood vessels, inducing proliferation of smooth muscle cells during atherogenesis, acting to increase cell spreading, inhibiting chemotaxis, and effecting the metabolism of lipoproteins and nonthrombogenic characteristics of endothelial cells. Additionally, it is believed that the HSPGs have different functions in different locations. For example, while cell surface HSPGs function as co-receptors for growth factors and support cell growth, extracellular HSPG can inhibit cell growth.

Although it is currently believed that endothelial HSPGs inhibit SMC proliferation, it is not known whether SMC synthesize antiproliferative HSPGs that act as autocrine inhibitors. Not wishing to be bound by any particular mechanism, it is currently believed that HSPGs inhibit DNA synthesis and SMC proliferation, and blocking HSPGs results in stimulation of DNA synthesis even in the absence of serum and growth factors. Known antiproliferative agents fail to inhibit SMC proliferation when the effects of HSPGs are blocked.

Examples of HSPGs include syndecan, glypican and perlecan which are HSPGs generated within the cardiovascular system. Vascular SMCs express syndecans 1, 2 and 4, glypican-1 and perlecan. The regulation of HSPG expression in these cells is not known. Cell growth stimulators such as platelet derived growth factor (PDGF), thrombin, serum, oxidized low density lipoproteins (LDL) and lysolecithin have been shown to decrease HSPG, and in particular, to decrease perlecan. In contrast, cellular antiproliferative agents, TGF-β, apolipoprotein E and heparin stimulate HSPGs.

The present invention comprises methods and compositions comprising the identification of compounds for the treatment and prevention of smooth muscle cell proliferation, preferably compounds that effect synthesis of proteoglycans (PG). More preferred embodiments comprise methods for screening for compounds or molecules that induce HSPG synthesis comprising the addition of such compounds to assays and measuring HSPG synthesis, including, but not limited to, the production of syndecan, glypican and perlecan. Most preferred embodiments comprise methods for measuring the induction of perlecan synthesis. HSPG production is important in regulating SMC proliferation, and the methods and compositions described herein provide for high throughput screening of molecules that induce HSPG production and regulate SMC proliferation.

A major extracellular HSPG in the blood vessel matrix is perlecan, a protein originally identified in basement membrane. It interacts with extracellular matrix proteins, growth factors and receptors. Perlecan is also present in basement membranes other than blood vessels and in other extracellular matrix structures. It consists of a core protein of Mr.˜450,000 to which three HS chains of Mr˜70 kDa are attached to one end of the molecule. Perlecan core protein has a complex functional organization consisting of five consecutive domains with homologies to molecules involved in control of cell proliferation, lipoprotein binding and cell adhesion. The N-terminal domain I (aa ˜1-195) contains attachment sites for HS chains. Domain II comprises four repeats homologous to the ligand-binding portion of the LDL receptor. Domain III has homology to domains IVa and IVb of laminin and is thought to mediate cell attachment.

Though not wishing to be bound by any particular mechanism, it is believed that extracellular HSPGs mediate quiescence in SMCs. In serum-starved quiescent SMC, perlecan synthesis is induced. For example, perlecan inhibits DNA synthesis and SMC proliferation, and blocking perlecan results in stimulation of DNA synthesis even in the absence of serum and growth factors. Induction of perlecan and other HSPGs is an important event for the inhibition of SMC growth. Known antiproliferative agents fail to inhibit SMC proliferation when the effects of perlecan are blocked. Thus, the present invention comprises methods and compositions for mediating perlecan and other HSPG synthesis, expression and amounts are taught for the maintenance of SMC in a quiescent state. Such methods and compositions of the present invention also comprise treatment and prevention of pathologies related to SMC proliferation. In particular, such pathologies include atherosclerosis and restenosis.

As used herein, the term “compound” includes both the singular and the plural, and includes any single entity or combined entities that have activity that can be measured in the assays of the present invention. Such entities include, but are not limited to, chemical elements, molecules, compounds, mixtures, emulsions, chemotherapeutic agents, pharmacological agents, hormones, antibodies, growth factors, cellular factors, nucleic acids, proteins, peptides, peptidomimetics, nucleotides, carbohydrates, and combinations, fragments, analogs or derivatives of such entities.

In the assays of the present invention, the compound initially has unknown activity, effect or effects. The activity of the compound is unknown, in that the compound's effects in the assays of the present invention are not yet determined. The compound may have many other known activities, and may be a compound that has other therapeutic uses. Any agent that causes the cells or components of the assay to respond in a measurable manner is contemplated by the present invention.

The present invention comprises methods and compositions for measuring the activity of unknown compounds. Such methods comprise assays for specific activity of biological components involved in a known cellular response. The assays provide a measurable response in which the activity of the unknown compounds is determined. This response can be measured by methods known to those skilled in the art, preferably in an ELISA. A preferred embodiment of the present invention comprises measurement of the effects of compounds on SMC proliferation in response to an HSPG-inducing agent.

In another embodiment of the present invention, a compound suspected of effecting HSPG synthesis is added to cells in an assay. The response of the cells can be measured by determining levels of HSPG synthesis measured by methods known to those skilled in the art and compared to the amount of HSPG synthesis in untreated cells. The compound may have a stimulating effect, an inhibitory effect, a stabilizing effect, or no effect at all.

In a further embodiment of the present invention, a composition suspected of effecting SMC proliferation is added to smooth muscle cells in growth medium or serum-free medium. The change in cell proliferation can be measured by methods known to those skilled in the art and compared to the proliferation of cells which are not treated with the compound. The composition may have a stimulating effect, an inhibitory effect, a stabilizing effect, or no effect at all.

The present invention also comprises compositions comprising the compounds identified by the methods as having a desired activity. The compositions have utility in treatment of cells, tissues or whole organisms. The compositions may be administered to humans and animals in dosages ranging from 0.001 mg to 200 mg/kg, preferably 0.001 mg to 100 mg/kg, more preferably 0.001 mg to 50 mg/kg, and most preferably 0.001 mg to 10 mg/kg. Such compositions are formulated for use in methods of administration in an effective amount for treatment of conditions such as biological conditions including, but not limited to, vascular occlusive lesions including atherosclerosis, transplant vasculopathy, cardiac allograft vasculopathy, restenosis, and graft atherosclerosis after coronary transplantation. The compositions may comprise other compounds including compounds with activities and pharmaceutical adjuncts that are needed for administration of the compound or compounds with the desired activity. The compositions may additionally be administered exclusively or in conjunction with other pharmaceutical compositions and surgical methods for treating smooth muscle cell proliferation and vascular occlusive diseases, including, but not limited to, before, during and after PTCA procedures.

The present invention comprises methods and compositions for the treatment and prevention of smooth muscle cell proliferation, including vascular occlusive pathologies. Such methods comprise administration of compositions comprising therapeutic agents capable of inhibiting SMC proliferation, preferably compositions that induce HSPGs such as syndecan, glypican and perlecan, and more preferably, compositions that induce perlecan synthesis and expression or effect the amount of perlecan. Such therapeutic agents that are effective in inducing HSPG activity or expression, preferably that of perlecan, are administered to animals suspected of having or who have vasculopathy or who have undergone angioplasty. Effective amounts of compositions are administered to such animals in dosages that are safe and effective.

Additionally, the present invention comprises methods and compositions for gene therapy, comprising administering compositions comprising nucleic acids that effect the synthesis or expression of HSPG, particularly perlecan. For example, vectors comprising nucleic acids coding for perlecan or active fragments of perlecan are provided to cells, preferably circulatory tissue cells, more preferably, endothelial cells. Such vectors are known to those skilled in the art and can be administered in formulations that enhance the uptake of the vector by the cells.

The present invention also comprises methods and compositions for inducing the synthesis or expression of HSPGs, including, but not limited to HSPGs such as syndecan, glypican and perlecan, preferably perlecan and also comprises induction and synthesis of active fragments of HSPGs, and more preferably, active fragments of perlecan. As used herein, when an HSPG is referred to, the entire molecule or fragments are included therein. For example, perlecan refers to the entire perlecan molecule or fragments thereof. Fragments of perlecan may have the same or different effects on cells. All of these fragments and activities are contemplated in the present invention.

The present invention additionally comprises diagnostic methods and compositions comprising detection of HSPG levels in the body, particularly levels of perlecan in the body, tissues or cells of a subject. Such diagnostic methods comprise measurement of HSPGs or HSPG fragment amounts, using methods disclosed herein or other methods known to those skilled in the art for measuring protein or nucleic acid levels. Changes in HSPG amounts or levels, and particularly, perlecan amounts or levels indicates changes in the proliferation levels of smooth muscle cells. Such diagnostic methods can be used to diagnose such changes in smooth muscle cells or to determine the effects of therapeutic agents or other therapies in a subject. For example, gene therapy methods are used to administer nucleic acids that effect perlecan synthesis or expression. Protein measurements, such as ELISA using monoclonal antibodies to perlecan, are made after administration to determine the therapeutic effect of the gene therapy. Positive increases in the levels of perlecan found after therapy indicates that the smooth muscle cell proliferation has ceased or will not be initiated.

The present invention further comprises methods and compositions for detecting the early stages of vascular occlusive conditions or other smooth muscle cell proliferation by determining the presence, amount or activity of HSPGs involved in SMC proliferation, particularly perlecan. Such methods and compositions comprise detection and quantification of perlecan using protein purification methods, immunoassays, nucleic acid measurements and other assays known to those in the art.

The present invention comprises methods and compositions for determining therapeutic agents that are capable of effecting SMC proliferation. Such assays are taught herein and can be used as assays to determine agents that effect the amount or activity of HSPGs, preferably perlecan, in such assays. For example, in one assay, perlecan is induced in cells by certain inducers, and the response is measured. Potential therapeutic agents are then added to a replicate assay and the effect on perlecan induction is determined. Using such methods and compositions, therapeutic agents are determined that can either inhibit, elevate induction of perlecan, or that have no effect at all. Such therapeutic agents can then be used in animals with SMC proliferation pathologies.

The compositions of the present invention may be administered through routes of administration that include, but are not limited to, oral, buccal, nasal, aerosol, topical, transdermal, injectable, slow release, controlled release, iontophoresis, sonophoresis, and other delivery devices and methods. Injectable methods include, but are not limited to, intravenous, intramuscular, intraperitoneal, intraspinal, intrathecal, intracerebroventricular, intraarterial, subcutaneous and intranasal routes.

The compositions for treating the pathologies by the present invention can further include a pharmaceutically acceptable carrier. The compositions can also include other medicinal agents, pharmaceutical agents, carriers, adjuvants diluents and other pharmaceutical preparations known to those skilled in the art. These agents are known to those skilled in the art and are generally described as being biologically inactive and can be administered to patients without causing deleterious interactions with the active agent. Examples of carriers or excipients for oral administration include corn starch, lactose, magnesium stearate, microcrystalline cellulose and stearic acid, povidone, dibasic calcium phosphate and sodium starch glycolate. Any carrier suitable for the desired administration route is contemplated by the present invention.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All patents and patent applications disclosed herein are hereby incorporated by reference in their entireties. All references listed or cited herein are incorporated by reference in their entireties.

The foregoing description includes the best presently contemplated mode of carrying out the invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

The Examples provided herein demonstrate that perlecan is an autocrine inhibitor of SMC growth. Perlecan expression is induced during serum starvation of cells in vitro. Purified perlecan inhibited SMC growth in cells provided with serum. Blocking perlecan either by an anti-perlecan antibody or by blocking perlecan synthesis abrogated the SMC quiescence induced in serum-free conditions. Finally, other antiproliferative agents such as cGMP, nitric oxide and glucosamine failed to inhibit SMC growth when perlecan was blocked.

### EXAMPLES

### Example I

Perlecan induction

Smooth muscle cells reach quiescence during serum starvation resulting in a blockade of DNA synthesis. To demonstrate perlecan's role in SMC quiescence, cells were starved by removing serum from the media. The cells used in this Example and the other examples herein were human aortic SMC, grown in basal medium supplemented with growth factors, bFGF and epidermal growth factor (EGF) (Clonetics, San Diego, Calif.).

SMC secretion of total PGs as well as perlecan were determined. PGs were radiolabeled with (³⁵S)sulfate by incubating the cells with (³⁵S)sulfate for 2 to 6 hours. Media PGs were collected and purified by DEAE-cellulose chromatography. Cell-associated PGs were assessed by extracting cells with 50 mM Tris buffer pH 7.4 containing 4 M urea, 1% Triton X-100, 0.1 mM EDTA and 1 mM PMSF. Aqueous solutions of (³⁵S)sulfate and (³H)leucine were from Amersham.

To determine changes in PG levels, DEAE-cellulose chromatography was performed. A DEAE-cellulose column was equilibrated with 50 mM Tris buffer pH 7.4 containing 4 M urea, 0.1 M NaCl, 0.1 mM EDTA, 1 mM PMSF and 1% 3[(3-cholamidopropyl)dimethylammonio]-1-propanesulfonate (CHAPS). The column was washed with the same buffer and buffer containing 0.25 M NaCl and PG were eluted with the same buffer containing 0.5 M NaCl. Fractions containing radioactivity (³⁵SO₄) were pooled and dialyzed against MEM overnight and counted.

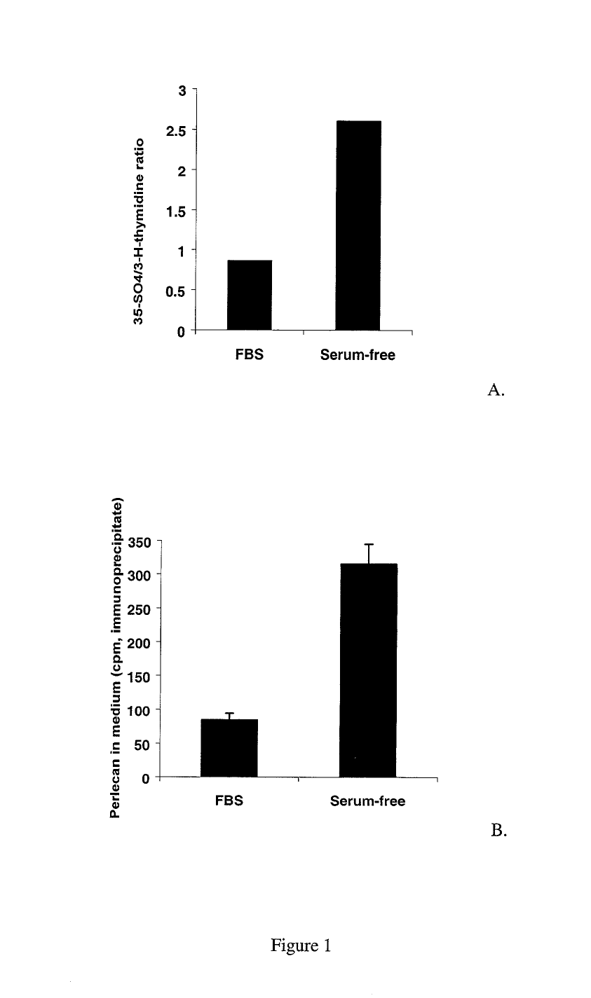

To determine the relative proportion of HSPG and chondroitin sulfate/dermatan sulfate proteoglycan (CS/DS PG), an aliquot of the pooled fraction was incubated in 50 mM sodium acetate buffer pH 5.2 with 1 unit/ml each of heparinase and heparitinase or with 0.5 units of chondroitin ABC lyase for 16 h at 37° C. Chondroitan ABC refers to different isomeric types of chondroitin, e.g. chondroitin A, chondroitin B, and chondroitin C. The reaction mixture was precipitated either with 0.5 volumes of 1% cetyl pyridinium chloride or with 3 volumes of ethanol to precipitate undigested glycosaminoglycans. Radioactivity in the supernatant and pellet was determined. (FIG. 1A)

To determine changes in perlecan protein, cells were grown in serum-free or serum-containing media in the presence of (³H)leucine for 24 h (steady state). Cells were plated at low density (8×10⁴/well in 48 well plate, 30-40% confluency) and cultured for 24 h (hour). Wells were then replenished with fresh medium containing no serum or 10% fetal bovine serum (FBS). Following another 24 h incubation, cells were labeled with (³H)thymidine for 6 h and radioactivity incorporated into the DNA was determined by trichloroacetic acid (TCA) precipitation of the cell lysate. (³H)thymidine was from NEN. Purified PG (0.5 M eluate) were immunoprecipitated by incubation with an anti-perlecan antibody (100-fold diluted) followed by precipitation with Protein A-Sepharose. Immunoprecipitates were analyzed by 5% SDS-PAGE. Perlecan (Mr>550 kDa) was identified by autoradiography. (FIG. 1B)

This example shows that serum starvation led to an increase in the secretion of ³⁵S-labeled proteoglycans into the medium. When expressed per cell, there was 2.5 fold increase in the amount of proteoglycans in media from serum-starved cells compared to media from cells grown in serum-containing media. To determine if serum starvation induces perlecan, perlecan was immunoprecipitated from media using anti-perlecan antibody. Similar to proteoglycan increase, media from serum-starved cells had a three-fold increase in perlecan. These data showed that serum-starvation induced perlecan expression by SMC.

### Example II

Perlecan inhibits smooth muscle cell proliferation

Although perlecan has been shown to negatively correlate with SMC growth, direct inhibitory effects of perlecan on SMC growth has never been previously demonstrated. Purified perlecan from SMC medium by DEAE-cellulose chromatography was obtained using methods in Example I, and was tested for its antiproliferative effects on SMC.

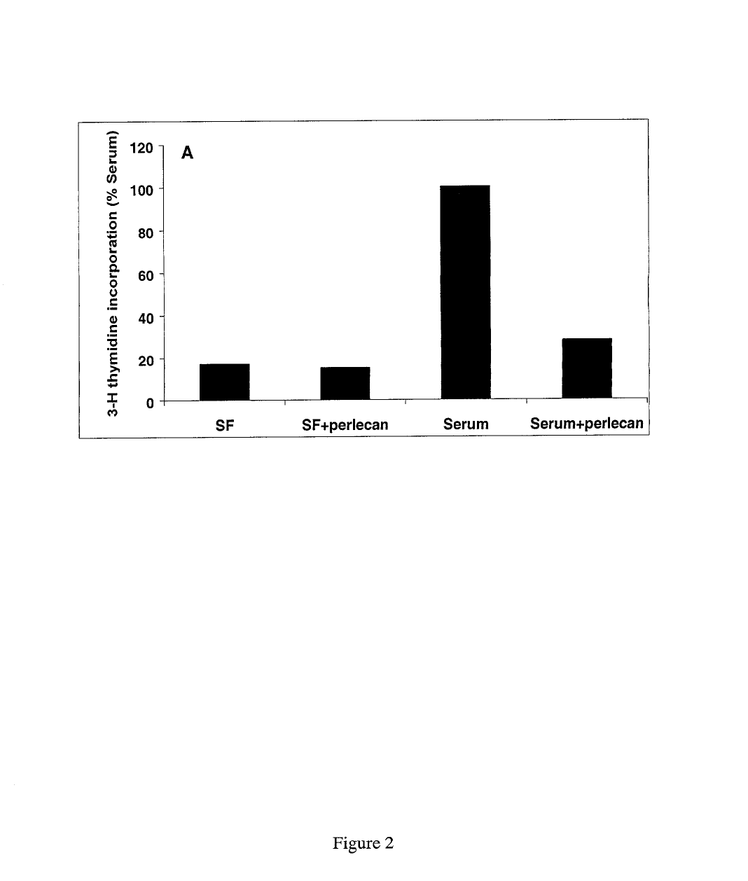

The addition of perlecan to serum-containing medium inhibited SMC growth by 70% (FIG. 2). Sub-confluent SMC (40-50% confluence) were incubated in serum-free medium or 10% serum-containing medium with or without purified perlecan for 24 h. DNA synthesis was then determined by incubating cells for another 5 h in medium containing (³H)thymidine. TCA precipitable (DNA) thymidine counts were determined and expressed as percentage of DNA synthesis in cells grown in 10%FBS.

### Example III

Perlecan mediates smooth muscle cell quiescence

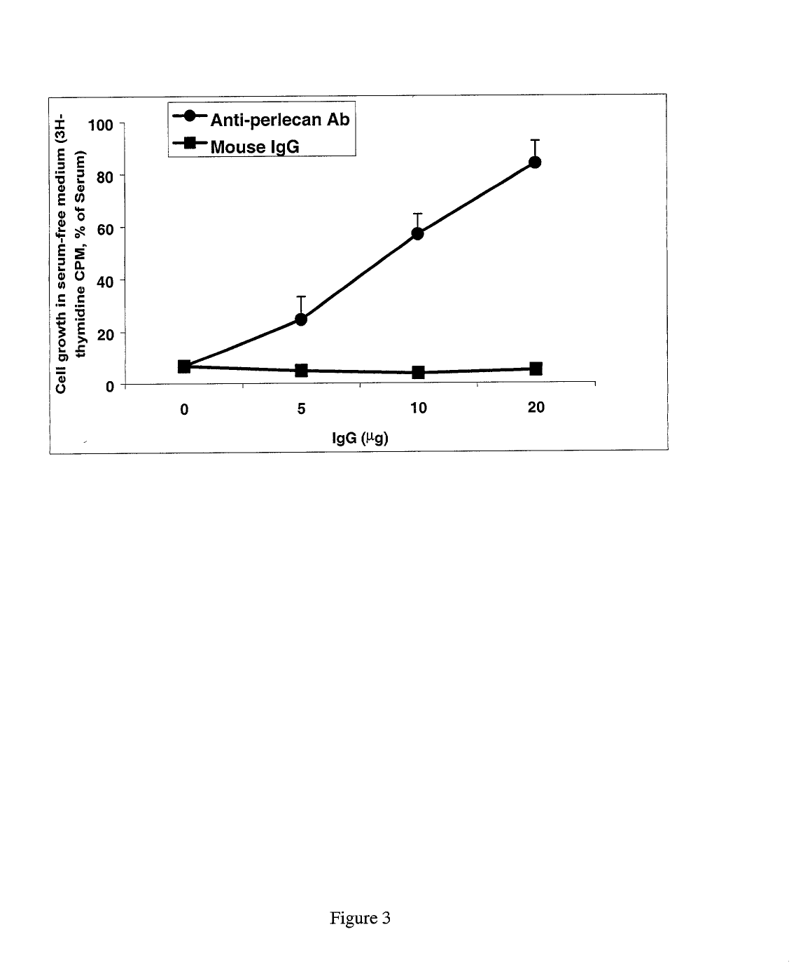

Perlecan, induced in serum-free conditions, was required for the blockade of DNA synthesis observed in serum-free conditions. The anti-perlecan antibody of Example I was used to block perlecan. Sub-confluent levels of SMC (40-50% confluence) were incubated in serum-free medium containing 0, 5, 10 and 20 μg of mouse IgG or anti-perlecan IgG for 24 hours. DNA synthesis was then determined by incubating cells for another 5 hours in medium containing (³H)thymidine. TCA precipitable (DNA) (³H)thymidine counts were determined and expressed as percentage of DNA synthesis in cells grown in 10% FBS. (FIG. 3) At 20-μg/ml concentration, anti-perlecan antibody restored DNA synthesis to near serum levels. Cells grown in serum-free conditions had a 80-90% decrease in DNA synthesis suggesting that cells have reached quiescence under these conditions. Addition of anti-perlecan IgG but not mouse IgG led to an increase in DNA synthesis. These data suggested that the quiescence caused by serum-free conditions is mediated by perlecan and that blocking perlecan removes the blockade on DNA synthesis.

### Example IV

Blocking perlecan synthesis leads to SMC growth in serum-free medium

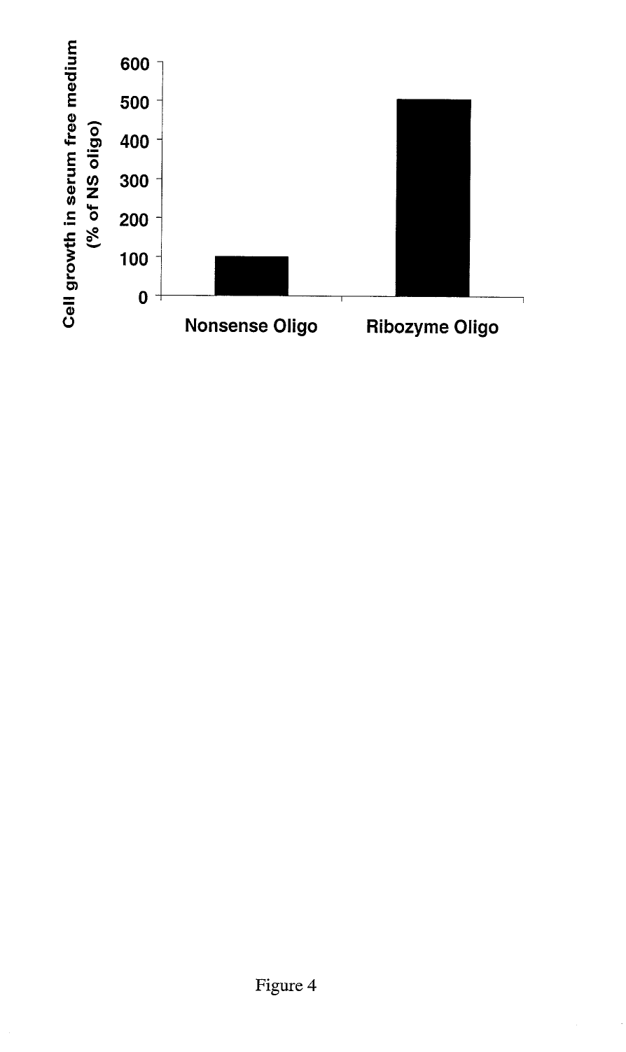

To further confirm the role of perlecan in SMC growth, SMC were transfected with a deoxyribozyme that is designed to cut perlecan MRNA at the mRNA start codon resulting in inhibition of perlecan production. The growth of SMC transfected with a non-specific oligonucleotide was compared to SMC transfected with perlecan-deoxyribozyme in serum-free conditions. These data are shown in FIG. 4.

Sub-confluent SMC (40-50% confluence) were transfected with a non-specific oligonucleotide or a deoxyribozyme oligonucleotide designed to cut perlecan niRNA. Following transfection, cells were incubated in serum-free medium for 24 h. DNA synthesis was then determined by incubating the cells for another 5 h in medium containing (3H)thymidine. TCA precipitable (DNA) thymidine counts were determined and expressed as a percentage of DNA synthesis in cells grown in 10% FBS.

Addition of the deoxyribozyme to interfere with perlecan synthesis did not affect growth in serum-containing medium. In serum-free conditions, however, blocking perlecan synthesis led to a five-fold increase in SMC DNA synthesis. These data further showed that perlecan was required to maintain SMC quiescence and that blocking perlecan synthesis lead to induction of DNA synthesis.

### Example V

Perlecan is required to mediate the effects of different antiproliferative agents

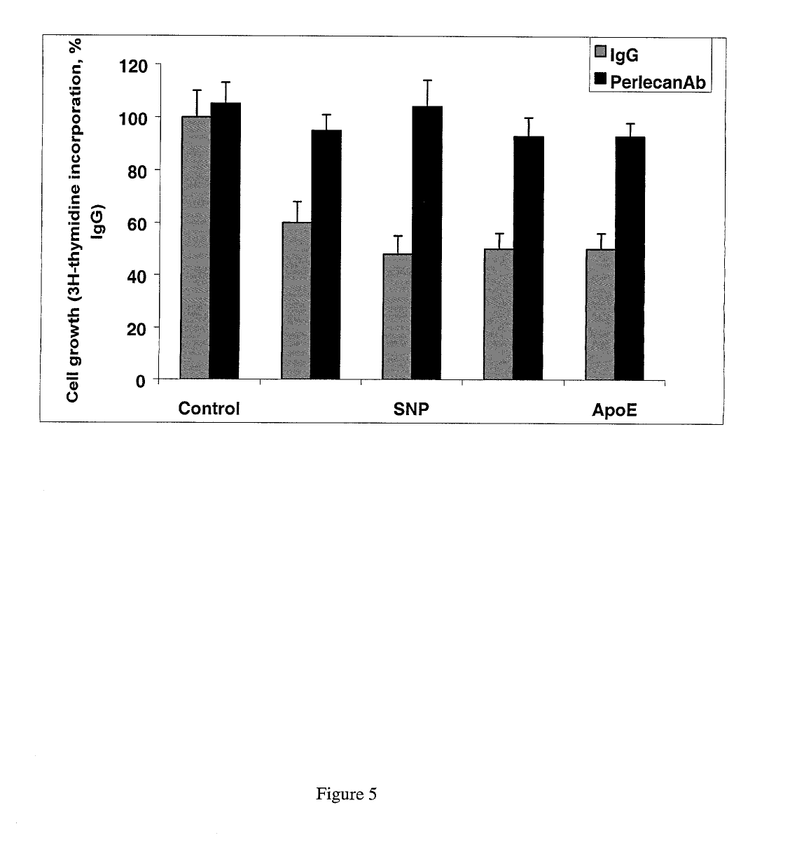

Known SMC growth inhibitors, 8 bromo cyclic GMP, nitric oxide donor sodium nitroprusside, glucosamine and apoE (20-21) were used in assays of the present invention. ApoE3 was purchased from Calbiochem (La Jolla, Calif.). These agents inhibited SMC proliferation by 50-60% (FIG. 5).

Sub-confluent SMC (40-50% confluence) were incubated in growth medium with or without different antiproliferative agents and with mouse IgG or with perlecan antibody for 24 hours. DNA synthesis was then determined by incubating cells for another 5 hours in medium containing (³H)thymidine. TCA precipitable (DNA) thymidine counts were determined and expressed as percentage of DNA synthesis in cells grown in 10% FBS.

Blockade of perlecan effect by anti-perlecan antibody completely abrogated the antiproliferative effects of these agents. These data showed that perlecan was an intermediate in the regulation of SMC growth.

### Example VI

High-throughput screening of compounds that effect perlecan synthesis.

Cells were initially plated at low density (8×104/well in 48 well plate, 30-40% confluency) and cultured for 24 h. To determine changes in perlecan protein, cells were grown in serum-free or serum-containing media, or cells were grown in serum-free media with a test compound, or serum-containing media with a test compound in the presence of (³H)leucine for 24 h (steady state). Wells then replenished with fresh medium containing no serum, no serum and a test compound, 10% FBS or 10% FBS and a test compound. Following another 24 h incubation, cells are labeled with (³H)thymidine for 6 h and radioactivity incorporated into the DNA is determined by TCA precipitation of the cell lysate. (³H)thymidine is from NEN.

10 μg each of test compounds 1-7 are added to cells and cultured as described above. HSPGs are isolated from SMC medium and purified by DEAE-cellulose chromatography. Compound 4 inhibits perlecan production. Compound 3 stimulates perlecan production and the remaining five compounds have no effect. Although only seven compounds are used, the method of the present invention is designed for high-throughput screening.

### Example VII

Evaluation of test compounds in vivo

Two of test compounds discovered using the methods of the present invention, particularly shown in Example VI, compositions RUS 3052 and RUS 3108, were evaluated for efficacy in the reduction of neointima formation following balloon catheter-induced injury of the common carotid arteries of male Wistar rats. Thirty-two rats were divided into groups of eight animals per group: two test compound groups and two control groups. The two control groups were the positive control, 5 mg/kg probucol, and the negative control, the vehicle for the test compositions and the positive control probucol. Probucol has been shown to reduce post-angioplasty restenosis in clinical trials and has been reported as effective in the rat balloon injury model. The vehicle solution was 10% DMSO, 10% cremophor, 20% propylene glycol in water for injection. Test compositions RUS 3052 and RUS 3108 were initially administered at 5 mg/kg up to and including the day of surgery. Starting the day after surgery, until the end of the study, the dosage of all compositions was lowered to 1 mg/kg. The test compositions were prepared by combining 5 mg of the test compound or control with 100 μl DMSO, 100 μl cremophor EL, and 200 μl propylene glycol with intermittent mixing using a cyclomixer. Water for injection was added in 100 μl aliquots to give a final volume of 1 ml.

The test compositions and controls were administered daily by intraperitoneal injection starting 72 hours prior to the surgical procedure and continued until necropsy, 14 days after the procedure. One animal from each of the four groups underwent the surgical procedure each day using a #2F Fogarty embolectomy catheter. An embolectomy catheter was introduced into the right external carotid artery and retrogradely passed to the aortic arch through the common carotid artery. The balloon was inflated with sterile saline and then pulled to the common carotid-external carotid bifurcation. Upon deflation, the procedure was repeated a second time. The catheter was removed and the proximal and distal ligatures to the arteriotomy of the external carotid were sutured.

### Example VIII

Efficacy of Test Compositions

Fourteen days post-surgery, the animals of Example VII were euthanized and the damaged right carotid arteries were collected, sectioned and stained with modified elastin-trichrome, and then analyzed for neointima formation that was quantified by histomorphometric analysis. In addition, sections from each damaged carotid artery were stained with hematoxylin-eosin and reviewed for histopathological changes. The undamaged left carotids were collected for immunohistochemistry analysis of the presence of perlecan. Animals were dosed the day of necropsy and blood samples were collected at necropsy.

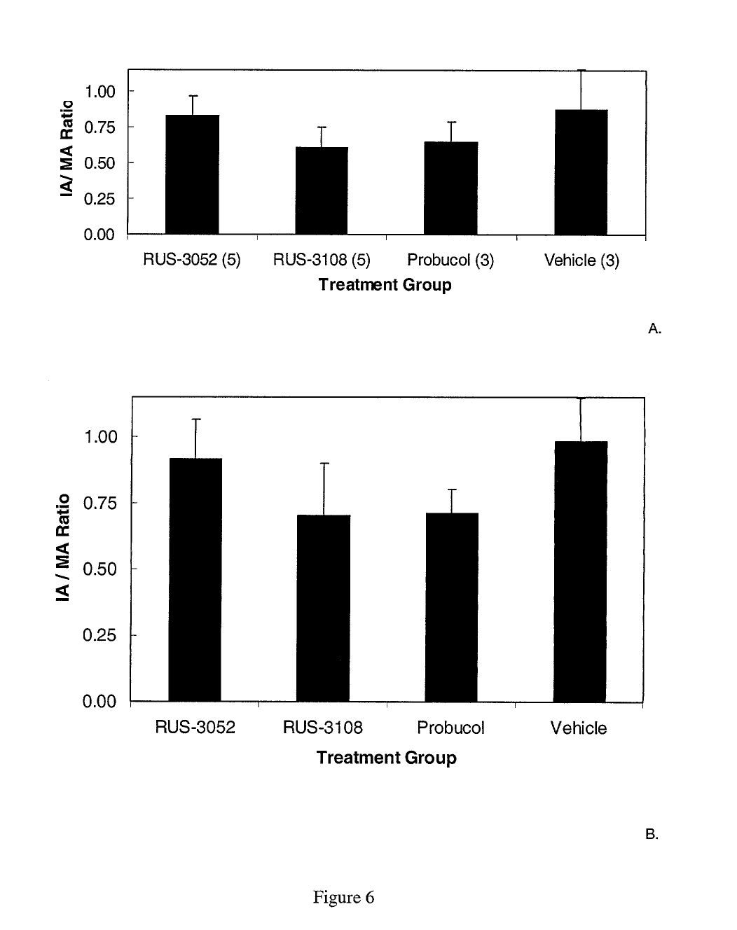

The ratio of intima area/media area (IA/MA) for each section of carotid artery that had neointima formation was reported based on histomorphometric results. The average IA/MA ratio was determined for each animal from all the sections analyzed that had an IA/MA ratio greater than 0.0915. The average IA/MA ratios and standard deviations based on animal averages within a group suggests that there was less LA, and thus, less neointima formation for animals that received test article RUS 3108 and the positive control, probucol. The results are shown in Table 1 and FIG. 6. FIG. 6A is a graph of the group average is the average of IA/MA ratios for each animal. All animals used in the group average had two or more sections and the sections had IA/MA ratios greater than 0.0915.

TABLE 1
IA/MA Averages

| | Based on Animal | Based on Section |
|---|---|---|
| | Averages | Averages |
| | IA/MA Average ± | IA/MA Average ± |
| Treatment | STD (n) | STD (n) |
| RUS 3052 | 0.83 ± 0.32 (5) | 0.92 ± 0.39 (16) |
| RUS 3108 | 0.61 ± 0.31 (5) | 0.71 ± 0.44 (17) |
| (+) Control Probucol | 0.65 ± 0.25 (3) | 0.71 ± 0.30 (9) |
| (−) Control Vehicle | 0.88 ± 0.50 (3) | 0.99 ± 0.40 (9) |

By averaging all of the sections within a group, rather than animal averages, there was a larger number of samples to perform statistical analysis using the two-tailed Student's t-Test. The average IA/MA ratios based on the sections within a group are shown in Table I and FIG. 6B. Test compound RUS 3052 compared to vehicle had a p value of 0.68, RUS 3108 compared to vehicle had a p value of 0.13, probucol compared to vehicle had a p value of 0.12. With significance set at p<0.20, these results support that RUS 3108 and probucol were effective in significantly reducing neointima formation in comparison to the negative control group.

### REFERENCES

1. Orford J L, Selwyn A P, Ganz P, Popma J J, Rogers C. (2000) The comparative pathobiology of atherosclerosis and restenosis. Am. J. Cardiol. 86:6H-11H.

2. Bult H. (2000) Restenosis: a challenge for pharmacology. Trends Pharmacol Sci. 21:274-9.

3. Schwartz S M. (1997) Smooth muscle migration in atherosclerosis and restenosis. J Clin Invest. 100:S87-9.

4. Rosenberg, R. D., Shworak N. W., Liu J. Schwartz J. J., and Zhang L. (1997) Heparan sulfate proteoglycans of vascular system. J. Clin. Invest. 99:2062-70.

5. Karnovsky, M. J., Wright, T. C. Jr., Castellot, J. J. Jr, Choay, J., Lormeau, J. C., Petitou, M. (1989) Heparin, heparan sulfate, smooth muscle cells, and atherosclerosis. Ann. N Y Acad. Sci. 556, 268-81.

6. Engelberg, H. (1991) Heparin, non-heparin glycosaminoglycans, and heparinoids: an overview of their application in atherosclerosis. Semin. Thromb. Hemost. 17 Suppl. 1:5-8.

7. Bingley J A, Campbell J H, Hayward I P, Campbell G R. (1997) Inhibition of neointimal formation by natural heparan sulfate proteoglycans of the arterial wall. Ann N Y Acad Sci. 811:238-44.

8. Koyama, N, Kinsella, M. G, Wight, T. N, Hedin, U, Clowes, A. W. (1998) Heparan sulfate proteoglycans mediate a potent inhibitory signal for migration of vascular smooth muscle cells. Circ. Res. 83, 305-313.

9. Iozzo, R. V., Cohen I. R, Grassel, S, Murdoch A. D. (1994) The biology of perlecan. Biochem. J. 302, 625-639.

10. Fuji, N, Kaji, T, Akai, T, Koizumi, F. (1997) Thrombin reduces large heparan sulfate proteoglycan molecules in cultured vascular endothelial cell layers through inhibition of core protein synthesis. Thromb. Res. 88, 299-307.

11. Ramasamy, S., Lipke, D. W., Boissonneault, G. A., Guo, H., Hennig, B. (1996) Oxidized lipid-mediated alterations in proteoglycan metabolism in cultured pulmonary endothelial cells. Atherosclerosis 120, 199-208.

12. Pillarisetti, S., Obunike, J. C. and Goldberg, I. J. (1995) Lysolecithin-induced alterations of subendothelial HSPG increase monocyte binding to matrix. J. Biol. Chem. 270, 29760-29765.

13. Pillarisetti, S., S. Paka, J. Obunike, L. Berglund, I. Goldberg. (1997) Evidence that reduced HS promotes lipoprotein (a) retention by subendothelial matrix. J. Clin. Invest. 100, 867-874.

14. Brown, C. T., Nugent, M. A., Lau, F. W., Trinkaus-Randall, V. (1999) Characterization of proteoglycans synthesized by cultured corneal fibroblasts in response to transforming growth factor beta and fetal calf serum. J. Biol. Chem. 274, 7111-7119.

15. lozzo, R. V., Pillarisetti, J., Sarma, B., Murdoch, A. Danielson, K., Uitto, J. and Mauviel, A. (1997) Structural characterization of perlecan gene promoter. J. Biol. Chem. 272, 5219-5228.

16. Paka, S., Goldberg, I. J., Obunike, J. C. Choi, S. Y., Saxena, U., Goldberg, I. D. and Pillarisetti, S. (1999b) Perlecan mediates the antiproliferative effect of apolipoprotein E on smooth muscle cells. An underlying mechanism for the modulation of smooth muscle cell growth. J. Biol.

Chem. 274, 36403-36408.

17. Weiser, M. C., Belknap, J. K., Grieshaber, S. S., Kinsella, M. G., Majack, R. A. (1996) Developmental regulation of perlecan gene expression in aortic smooth muscle cells. Matrix Biol. 15, 331-340.

18. Paka, S., Obunike, J. C. Kako, Y. and Pillarisetti, S. (1999) Apo E stimulates endothelial production heparan sulfate rich in biologically active heparin-like domains. J. Biol. Chem.274, 4816-4823.

19. Belknap J K, Weiser-Evans M C, Grieshaber S S, Majack R A, Stenmark K R. (1999) Relationship between perlecan and tropoelastin gene expression and cell replication in the developing rat pulmonary vasculature. Am. J. Respir.

Cell Mol Biol. 20:24-34.

20. Garg U C, Hassid A. (1989) Nitric oxide-generating vasodilators and 8-bromo-cyclic guanosine monophosphate inhibit mitogenesis and proliferation of cultured rat vascular smooth muscle cells. J Clin Invest.83: 1774-7.

21. Yu S M, Hung L M, Lin C C. (1997) cGMP-elevating agents suppress proliferation of vascular smooth muscle cells by inhibiting the activation of epidermal growth factor signaling pathway. Circulation. 95:1269-77.

22. Campbell J H, Campbell G R. (1986) Endothelial cell influences on vascular smooth muscle phenotype. Annu. Rev. Physiol. .48:295-306.

23. Kamovsky M J. (1981) Endothelial——vascular smooth muscle cell interactions. Rous——Whipple Award Lecture.Am. J. Pathol. 105:200-6.

24. Benitz W E, Kelley R T, Anderson C M, Lorant D E, Bemfield M. (1990) Endothelial heparan sulfate proteoglycan. I. Inhibitory effects on smooth muscle cell proliferation. Am. J. Respir. Cell. Mol. Biol. 2:13-24.

25. Luscher T F, Noll G. (1995) The pathogenesis of cardiovascular disease: role of the endothelium as a target and mediator. Atherosclerosis.118 Suppl:S81-90.

26. Forsten K E, Courant N A, Nugent M A. (1997) Endothelial proteoglycans inhibit bFGF binding and mitogenesis. J Cell Physiol.172:209-20.

27. Ottlinger M E, Pukac L A, Karnovsky M J. (1993) Heparin inhibits mitogen-activated protein kinase activation in intact rat vascular smooth muscle cells. J Biol Chem. 268:19173-6.

28. Hayashi, K., Madri, J. A., Yurchenco, P. D. (1992) Endothelial cells interact with the core protein of basement membrane perlecan through beta 1 and beta 3 integrins: an adhesion modulated by glycosaminoglycan. J Cell Biol. 19,945-59.

29. Crowley S T, Ray C J, Nawaz D, Majack R A, Horwitz L D. (1995) Multiple growth factors are released from mechanically injured vascular smooth muscle cells. Am J Physiol. 269(5 Pt 2):H1641-7.

What is claimed is:

1. A method for detecting compounds that effect cell proliferation, comprising,
a) adding to cells a composition comprising a compound suspected of effecting cell proliferation;
b) measuring cell proliferation; and
c) comparing cell proliferation in b) to cell proliferation in cells not treated with the compound.

2. The method of claim 1, wherein cell proliferation is determined by measuring the amount of HSPG.

3. The method of claim 3, wherein the HSPG is perlecan, syndecan or glypican.

4. The method of claim 2, wherein the HSPG is perlecan.

5. The method of claim 1, wherein the compound is a chemical element, molecule, compound, mixture, emulsion, chemotherapeutic agent, pharmacological agent, hormone, antibody, growth factor, cellular factor, nucleic acid, protein, peptide, peptidomimetic, nucleotide, carbohydrate, and combinations, fragments, analogs or derivatives of such entities.

6. A composition, comprising, a compound capable of effecting cellular proliferation.

7. The composition of claim 6, wherein the compound increases HSPG production.

8. The composition of claim 6 wherein the compound decreases the production of HSPG.

9. The composition of claim 6, wherein the compound is a chemical element, molecule, compound, mixture, emulsion, chemotherapeutic agent, pharmacological agent, hormone, antibody, growth factor, cellular factor, nucleic acid, protein, peptide, peptidomimetic, nucleotide, carbohydrate, and combinations, fragments, analogs or derivatives of such entities.

10. The composition of claim 6, wherein the compound increases cell proliferation as determined by the method of claim 1.

11. The composition of claim 6, wherein the compound decreases cell proliferation as determined by the method of claim 1.

12. The composition of claim 6 wherein the composition comprises a nucleic acid vector.

13. The composition of claim 6, further comprising a pharmaceutically acceptable carrier.

14. A method for treating vascular occlusive conditions, comprising, administering a composition comprising one or more compounds in an effective amount for inducing HSPG synthesis.

15. The method of claim 14 wherein the HSPG is syndecan, glypican or perlecan.

16. The method of claim 14 wherein the vascular occlusive condition is atherosclerosis, thrombosis, transplant vasculopathy, cardiac allograft vasculopathy or restenosis.
